(19) United States
(12) Patent Application Publication
(10) Pub. No.: US 2003/0105315 A1
(43) Pub. Date: Jun. 5, 2003

(54) Human nucleic acid sequences from ovarian tumor tissue

(75) Inventors: Thomas Specht, Berlin (DE); Bernd Hinzmann, Berlin (DE); Armin Schmitt, Berlin (DE); Christian Pilarsky, Schonfeld (DE); Edgar Dahl, Potsdam (DE); Andre Rosenthal, Berlin (DE)

Correspondence Address: MILLEN WHITE ZELANO & BRANIGAN, P.C., Suite 1400, 2200 Clarendon Boulevard, Arlington, VA, 22201, US

(73) Assignee: metaGen Pharmaceuticals GmbH

(21) Appl. No.: 10/272,138

(22) Filed: Oct. 15, 2002

Related U.S. Application Data
(62) Division of application No. 09/646,778, filed on Sep. 22, 2000.

(30) Foreign Application Priority Data
Apr. 9, 1998 (DE) 198 17 557.4

(51) Int. Cl. C12Q 1/68; G01N 33/574; C07H 21/04; C12N 9/00

(52) U.S. Cl. 536/23.2; 435/6; 435/7.23; 435/183; 435/320.1; 435/325

(57) ABSTRACT

The invention relates to human nucleic acid sequences—mRNA, cDNA, genome sequences—of ovarian tumour tissue, which code for gene products or parts of these products, and to their use. The invention also relates to the polypeptides obtained by way of these sequences and to the use of same.

The invention relates to human nucleic acid sequences from ovarian tumor tissue, which code for gene products or parts thereof, their functional genes that code at least one bioactive polypeptide and their use.

In addition, the invention relates to the polypeptides that can be obtained by way of the sequences and their use.

One of the main cancer causes of death in women is ovarian cancer, for control of which new therapies are necessary. Previously used therapies such as, e.g., chemotherapy, hormone therapy or surgical removal of tumor tissue, frequently do not result in a complete cure.

The cancer phenomenon often goes along with overexpression or underexpression of certain genes in degenerated cells, it still being unclear whether these altered expression rates are the cause or the result of the malignant transformation. Identification of these genes would be an important step for development of new therapies against cancer. Spontaneous formation of cancer is often preceded by a host of mutations. They can have the most varied effects on the expression pattern in the affected tissue, such as, e.g., underexpression or overexpression, but also expression of shortened genes. Several such changes due to these mutation cascades can ultimately lead to malignant degeneration. The complexity of these relationships makes an experimental approach very difficult.

A database that consists of so-called ESTs is used to look for candidate genes, i.e., genes that compared to the tumor tissue are more strongly expressed in normal tissue. ESTs (expressed sequence tags) are sequences of cDNAs, i.e., mRNAs transcribed in reverse, therefore molecules that reflect gene expression. The EST sequences are determined for normal and degenerated tissue. These databases are offered to some extent commercially by various companies. The ESTs of the LifeSeq database, which is used here, are generally between 150 and 350 nucleotides long. They represent a pattern that is unmistakable for a certain gene, although this gene is normally very much longer (>2000 nucleotides). By comparison of the expression patterns of normal and tumor tissue, ESTs can be identified that are important for tumor formation and proliferation. There is, however, the following problem: Since the EST sequences that are found can belong to different regions of an unknown gene due to different constructions of cDNA libraries, in this case a completely incorrect ratio of the occurrence of these ESTs in the respective tissue would arise. This would only be noticed when the complete gene is known and thus ESTs can be assigned to the same gene.

It has now been found that this error possibility can be reduced if all ESTs from the respective tissue type are assembled beforehand, before the expression patterns are compared to one another. Overlapping ESTs of the same gene were thus combined into longer sequences (see FIG. 1, FIG. 2a and FIG. 3). This lengthening and thus coverage of an essentially larger gene region in each of the respective bases are intended to largely avoid the above-described error. Since there were no existing software products for this purpose, programs for assembling genomic sections were employed, which were used modified and to which our own programs were added. A flow chart of the assembly procedure is shown in FIGS. 2b1-2b4.

Nucleic acid sequence Seq. ID No 115, which plays a role as candidate gene in ovarian cancer, has now been found.

The invention thus relates to a nucleic acid sequence that codes a gene product or a part thereof, comprising

a) a nucleic acid sequence Seq. ID No. 115,

b) an allelic variation of the nucleic acid sequence named under a) or

c) a nucleic acid sequence that is complementary to the nucleic acid sequence named under a) or b).

The invention also relates to nucleic acid sequence Seq. ID No. 115, which is expressed elevated in ovarian tumor tissue.

The invention further relates to nucleic acid sequences comprising a portion of the above-mentioned nucleic acid sequence in such a sufficient amount that they hybridize with sequence Seq. ID No. 115.

The nucleic acid sequences according to the invention generally have a length of at least 50 to 4500 bp, preferably a length of at least 150 to 4000 bp, especially a length of 450 to 3500 bp.

With the partial sequence Seq. ID No. 115 according to the invention, expression cassettes can also be built using current process practice, whereby on the cassette at least one of the nucleic acid sequences according to the invention is combined with at least one control or regulatory sequence generally known to one skilled in the art, such as, e.g., a suitable promoter. The sequences according to the invention can be inserted in a sense or antisense orientation.

A large number of expression cassettes or vectors and promoters which can be used are known in the literature.

Expression cassettes or vectors are defined as: 1. bacterial, such as, e.g., phagescript, pBs, ΦX174, pbluescript SK, pBs KS, pNH8a, pNH16a, pNH18a, pNH46a (Stratagene), pTrc99A, pKK223-3, pKK233-3, pDR540, pRIT5 (Pharmacia), 2. eukaryotic, such as, e.g., pWLneo, pSV2cat, pOG44, pXT1, pSG (Stratagene), pSVK3, pBPV, pMSG, PSVL (Pharmacia).

A control or regulatory sequence is defined as suitable promoters. Here, two preferred vectors are the pKK232-8 and the PCM7 vector. In particular, the following promoters are intended: lacI, lacZ, T3, T7, gpt, lambda P_{R}, trc, CMV, HSV thymidine-kinase, SV40, LTRs from retrovirus and mouse metallothionein-I.

The DNA sequences located on the expression cassette can code a fusion protein which comprises a known protein and a bioactive polypeptide fragment.

The expression cassettes are likewise the subject matter of this invention.

The nucleic acid fragments according to the invention can be used to produce full-length genes. The genes that can be obtained are likewise the subject matter of this invention.

The invention also relates to the use of the nucleic acid sequences according to the invention and the gene fragments that can be obtained from use.

The nucleic acid sequences according to the invention can be moved with suitable vectors into host cells, in which as the heterologous part, the genetic information which is contained on the nucleic acid fragments and which is expressed is located.

The host cells containing the nucleic acid fragments are likewise the subject matter of this invention.

Suitable host cells are, e.g., prokaryotic cell systems such as E. coli or eukaryotic cell systems such as animal or human cells or yeasts.

The nucleic acid sequences according to the invention can be used in the sense or antisense form.

Production of polypeptides or their fragments is done by cultivation of the host cells according to current cultivation methods and subsequent isolation and purification of the peptides or fragments, likewise using current methods.

The invention further relates to nucleic acid sequences, which code at least a partial sequence of a bioactive polypeptide.

The invention also relates to antibodies that are directed ageinst a polypeptide or a fragment thereof and that are coded by the nucleic acid sequence Seq. ID No. 115. Antibodies are defined especially as monoclonal antibodies. Such antibodies can be identified by, i.a., a phage display process.

The polypeptide partial sequences coded by the sequences of the invention can be used in a phage display process. The polypeptides that are identified with this process and that bind to the polypeptide partial sequences coded by sequences according to the invention are also the subject matter of the invention.

The nucleic acid sequences according to the invention can also be used in a phage display process.

The invention also relates to phage-display phages, which are directed against a polypeptide or a fragment and which are coded by the nucleic acid of sequence Seq. ID No. 115 according to the invention.

The polypeptides according to the invention can also be used as tools for finding active ingredients against ovarian cancer, which is likewise the subject matter of this invention.

Likewise the subject matter of this invention is the use of nucleic acid sequences according to sequence Seq. ID No. 115 for expression of polypeptides, which can be used as tools for finding active ingredients against ovarian cancer.

The nucleic acid sequences found according to the invention can also be genomic or mRNA sequences.

The invention also relates to genomic genes, their exon and intron structures and their splice variants that can be obtained from cDNAs of sequence Seq. ID No. 115, and their use together with suitable regulatory elements, such as suitable promoters and/or enhancers.

With the nucleic acids according to the invention (cDNA sequences), genomic BAC, PAC and Cosmid libraries are screened and specifically human clones are isolated via complementary base pairing (hybridization). The BAC, PAC and Cosmid clones isolated in this way are hybridized using fluorescence-in-situ hybridization on metaphase chromosomes and the corresponding chromosome sections on which the corresponding genomic genes lie are identified. BAC, PAC and Cosmid clones are sequenced in order to clarify the corresponding genomic genes in their complete structure (promoters, enhancers, silencers, exons and introns). BAC, PAC and Cosmid clones can be used as independent molecules for gene transfer (see FIG. 5).

The invention also relates to BAC, PAC and Cosmid clones containing functional genes and their chromosomal localization according to sequence Seq. ID No. 115 for use as vehicles for gene transfer.

Meanings of Technical Terms and Abbreviations

| | |
|---|---|
| Nucleic acids | Nucleic acids in this invention are defined |
| | as: mRNA, partial cDNA, full-length cDNA |
| | and genomic genes (chromosomes). |
| ORF | Open Reading Frame, a defined sequence of |
| | amino acids which can be derived from the |
| | cDNA sequence. |
| Contig | A set of DNA sequences that can be combined |
| | as a result of very great similarities into |
| | one sequence (consensus). |
| Singleton | A contig that contains only one sequence. |
| Module | Domain of a protein with a defined se- |
| | quence, which represents one structural |
| | unit and which occurs in various proteins. |
| N | selectively the nucleotide A, T, G or C. |
| X | selectively one of the 20 naturally occur- |
| | ring amino acids. |

Explanation of the Alignment Parameters

minimal initial match=minimal initial identity area

maximum pads per read=maximum number of insertions

maximum percent mismatch=maximum deviation in %

### EXPLANATION OF FIGURES

FIG. 1 shows the systematic gene search in the Incyte LifeSeq database

FIG. 2a shows the principle of EST assembling

FIGS. 2b1-2b4 show the entire principle of EST assembling

FIG. 3 shows the in-silico subtraction of gene expression in various tissues

FIG. 4a shows the determination of tissue-specific expression via electronic Northern

FIG. 4b shows the electronic Northern

FIG. 5 shows the isolation of genomic BAC and PAC clones.

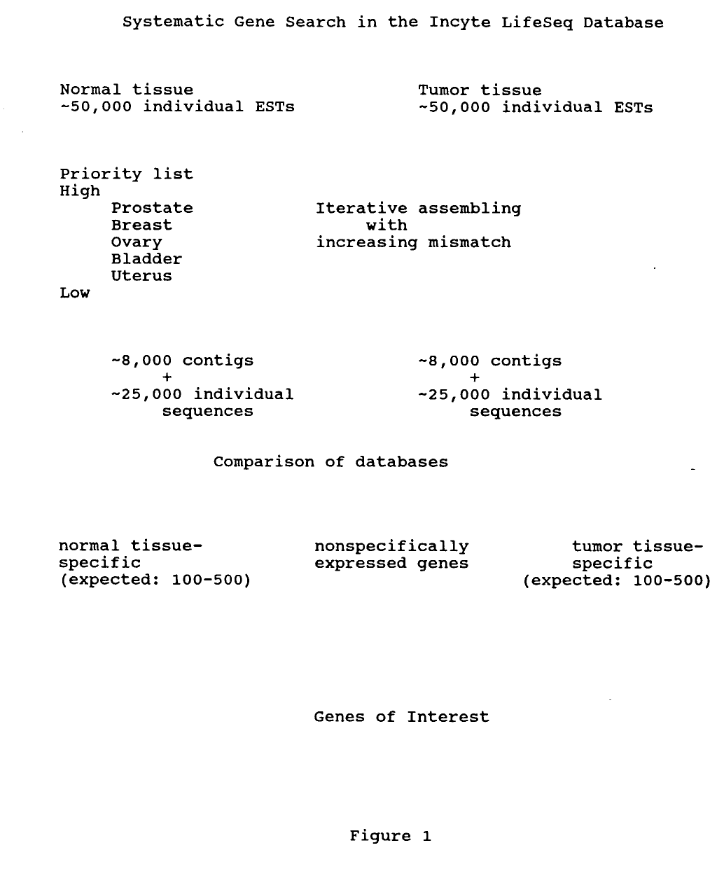

The following examples explain the production of the nucleic acid sequences according to the invention without limiting the invention to these examples and nucleic acid sequences.

### EXAMPLE 1

Search for Tumor-Related Candidate Genes

First, all ESTs of the corresponding tissue from the LifeSeq database (from October 1997) were extracted. They were then assembled by means of the GAP4 program of the Staden package with the parameters 0% mismatch, 8 pads per read and a minimal match of 20. The sequences (fails) not recorded in the GAP4 database were assembled first at 1% mismatch and then again at 2% mismatch with the database. Consensus sequences were computed from the contigs of the database that consisted of more than one sequence. The singletons of the database, which consisted of only one sequence, were re-assembled at 2% mismatch with the sequences not recorded in the GAP4 database. In turn, the consensus sequences were determined for the contigs. All other ESTs were reassembled at 4% mismatch. The consensus sequences were extracted once again and finally assembled with the previous consensus sequences and the singletons and the sequences not recorded in the database at 4% mismatch. The consensus sequences-were formed and used with the singletons and fails as the initial basis for tissue comparisons. This procedure ensured that among the parameters used, all sequences represented gene regions independent of one another.

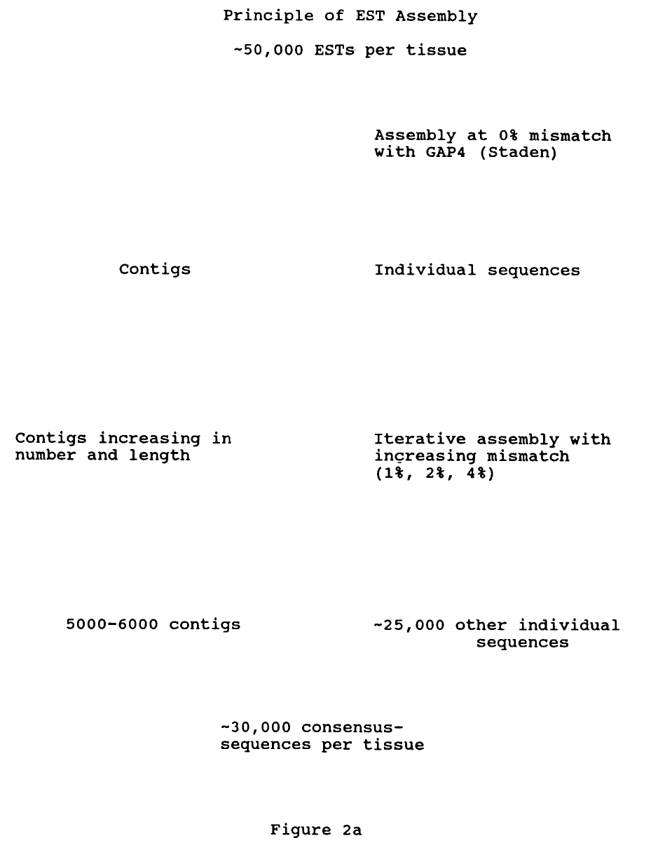

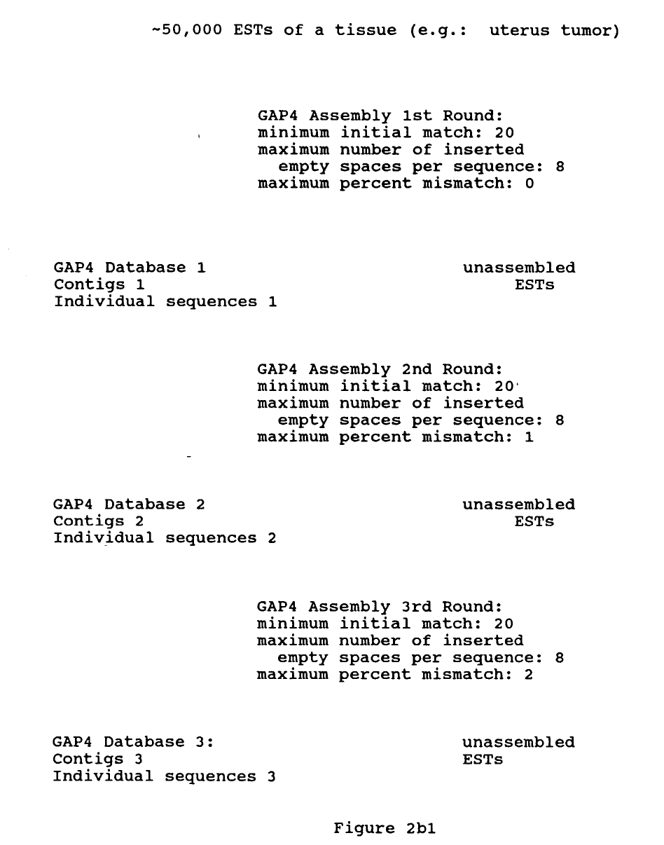

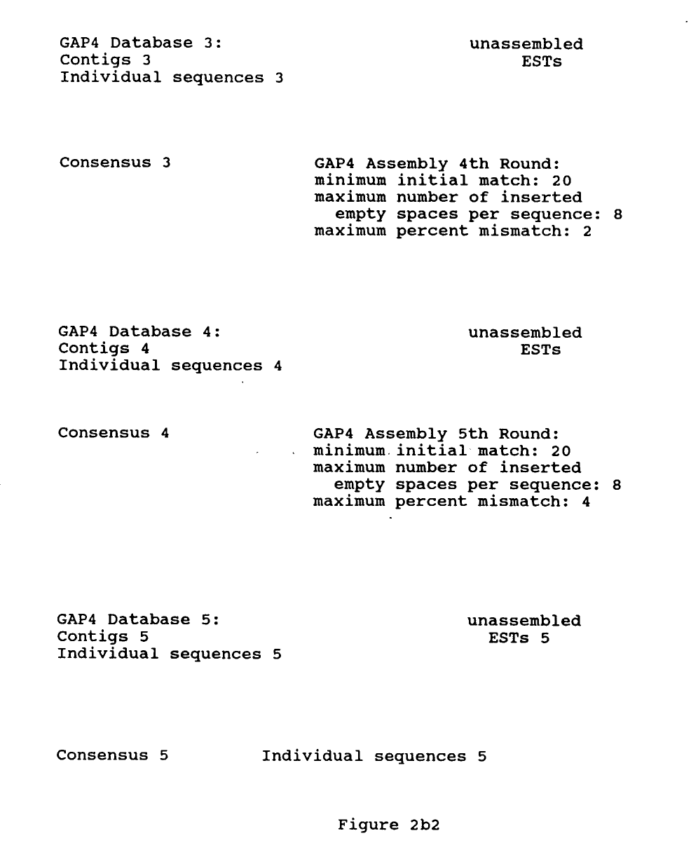

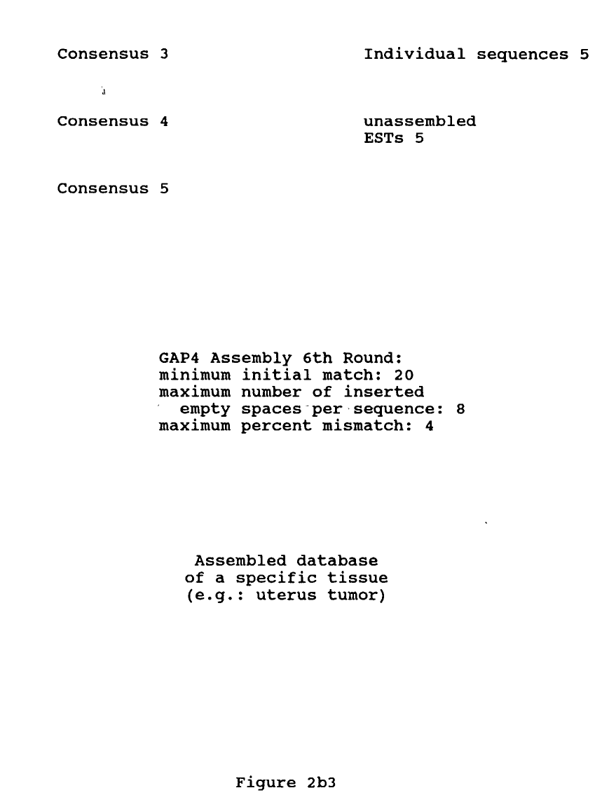

FIGS. 2b1-2b4 illustrate the lengthening of the ovarian tumor tissue ESTs.

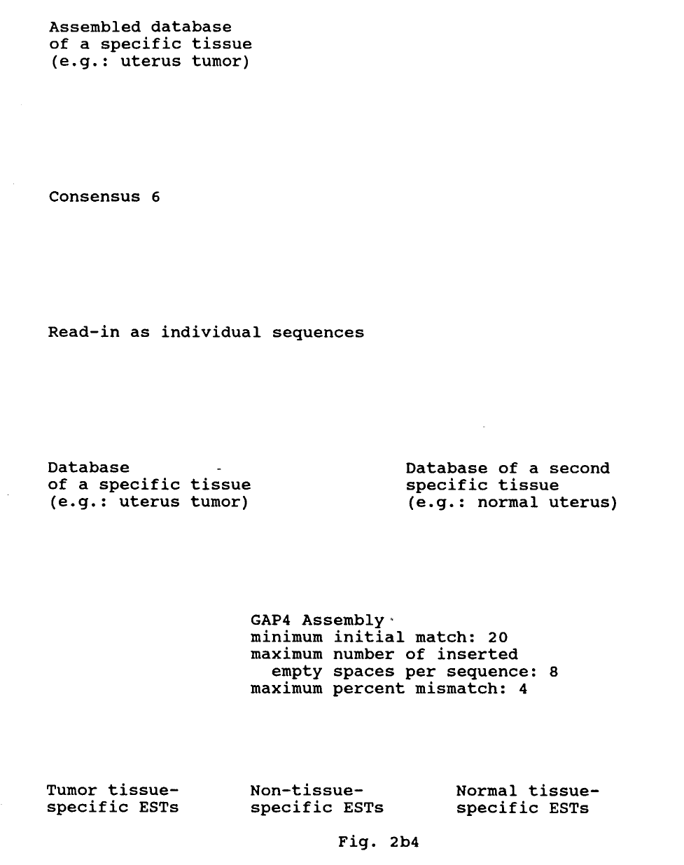

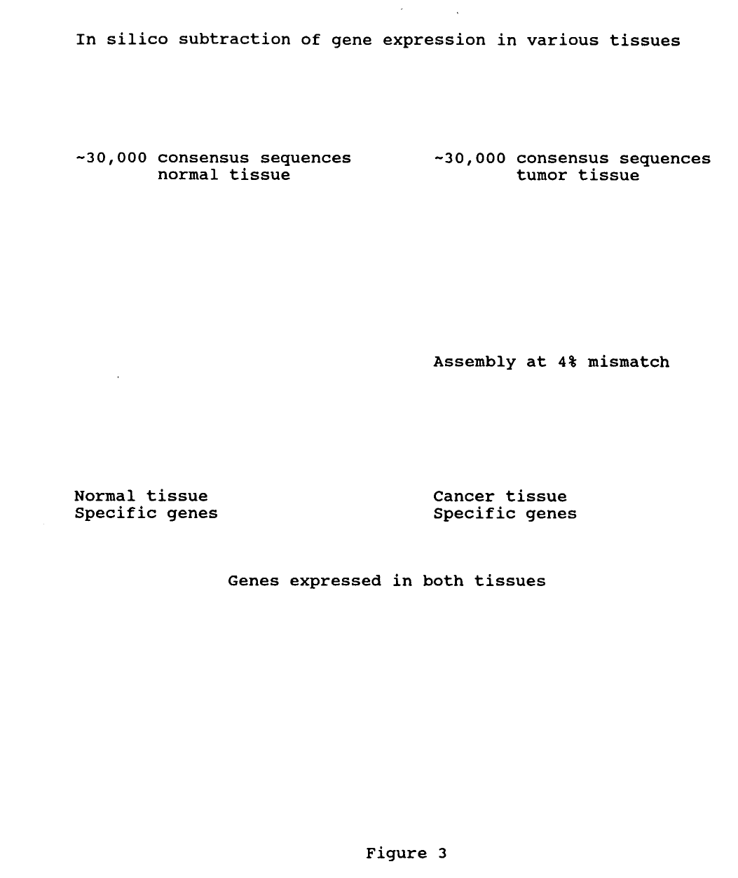

The sequences of the respective tissue assembled in this way were then compared to one another by means of the same program (FIG. 3). To do this, first all sequences of the first tissue were input into the database. (It was therefore important that they were independent of one another).

Then, all sequences of the second tissue were compared to all those of the first. The result was sequences that were specific to the first or the second tissue as well as those which occurred in both. In the latter, the ratio of the frequency of occurrence in the respective tissue was evaluated. All programs pertaining to the evaluation of the assembled sequences were themselves developed.

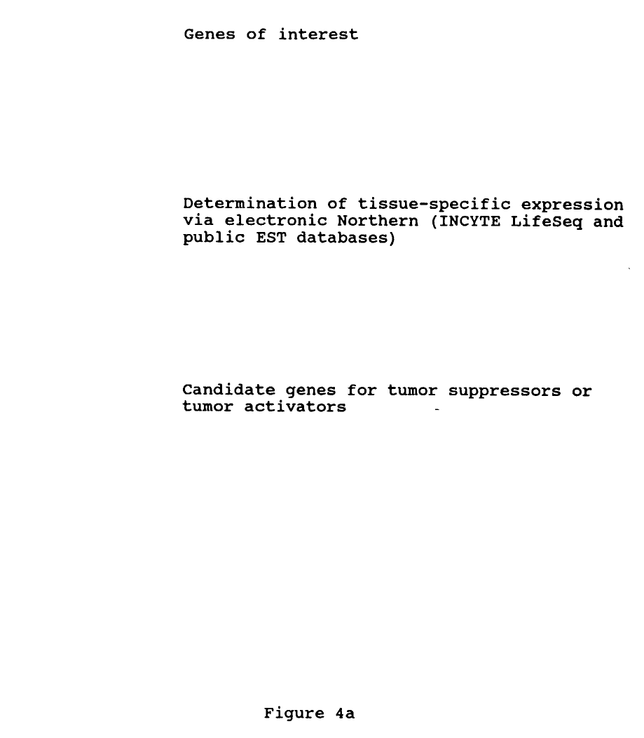

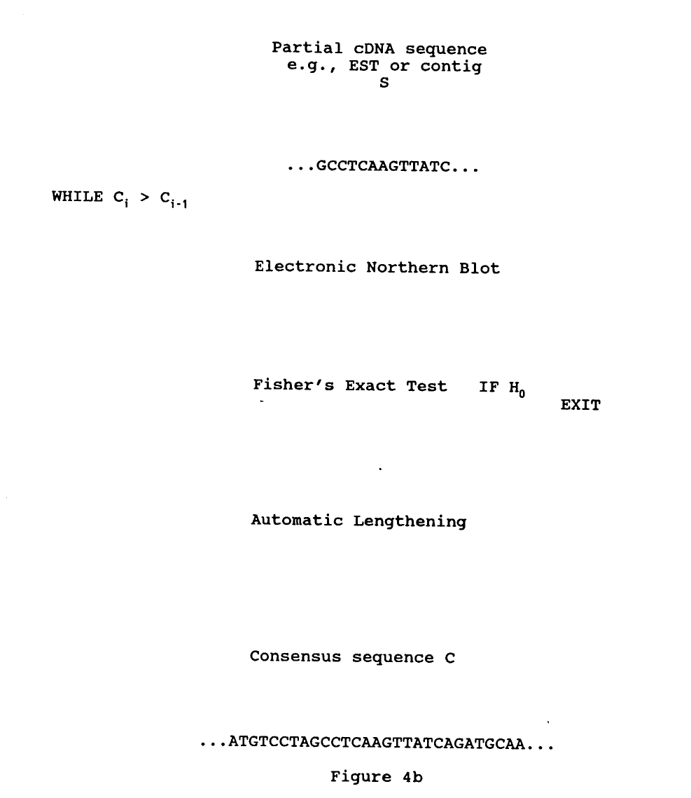

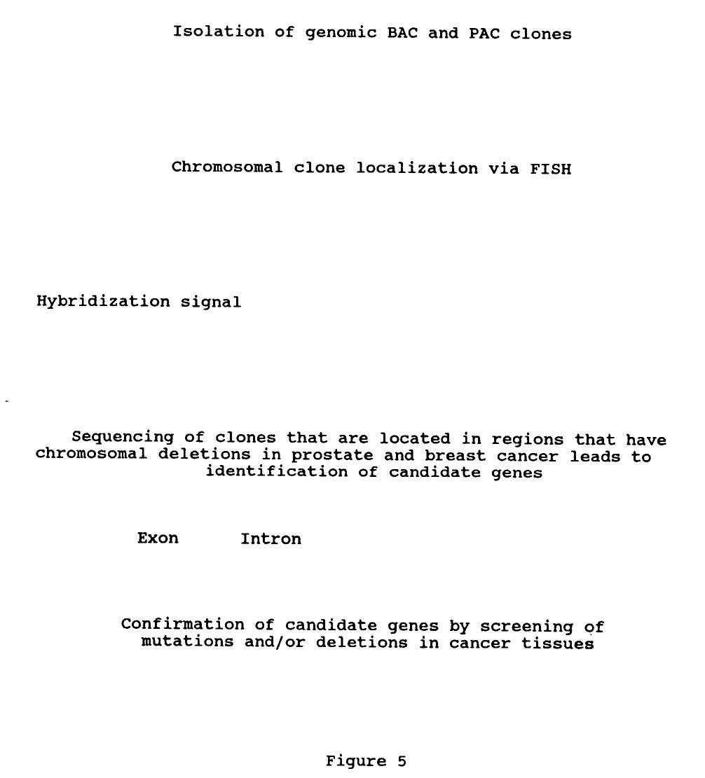

All sequences that occurred more than four times in respectively one of the compared tissues and all that occurred at least five times as often in one of the two tissues were further studied. These sequences were subjected to an electronic Northern (see Example 2.1), by which the distribution in all tumor and normal tissues was studied (see FIG. 4a and FIG. 4b). The relevant candidates were then lengthened using all Incyte ESTs and all ESTs of public databases (see Example 3). Then, the sequences and their translation into possible proteins were compared to all nucleotide and protein databases and were studied for possible regions that code for proteins.

### EXAMPLE 2

Algorithm for Identification and Lengthening of Partial EDNA Sequences with Altered Expression Pattern

An algorithm for finding overexpressed or underexpressed genes will be explained below. The individual steps are also summarized in a flow chart for the sake of clarity (see FIG. 4b).

2.1. Electronic Northern Blot

By means of a standard program for homology search, e.g., BLAST (Altschul, S. F.; Gish, W.; Miller, W.; Myers, E. W. andLipman, D. J. (1990) J. Mol. Biol. 215, 403-410), BLAST2 (Altschul, S. F.; Madden, T. L.; Sch@ffer, A. A.; Zhang, J.; Zhang, Z.; Miller, W., and Lipman, D. J. (1997) Nucleic Acids Research 25 3389-3402) or FASTA (Pearson, W. R. and Lipman, D. J. (1988) Proc. Natl. Acad. Sci. USA 85 2444-2448), the homologous sequences in various EST libraries (private or public) arranged by tissues are determined for a partial DNA sequence S, e.g., an individual EST or a contig of ESTS. The (relative or absolute) tissue-specific occurrence frequencies of this partial sequence S which were determined in this way are called electronic Northern Blots.

2.1.1

Analogously to the procedure described under 2.1, the sequence Seq. ID No. 115 was found. The result is given in Table 1.

2.2. Fisher Test

In order to decide whether a partial sequence S of a gene occurs significantly more often or less often in a library for normal tissue than in a library for degenerated tissue, Fisher's exact test, a standard statistical process, is carried out (Hays, W. L., (1991) Statistics, Harcourt Brace College Publishers, Fort Worth).

The null hypothesis reads: The two libraries cannot be distinguished with respect to the frequency of sequences homologous to S. If the null hypothesis can be rejected withhigh enough certainty, the gene belonging to S is accepted as an advantageous candidate for a cancer gene, and in the next step an attempt is made to achieve lengthening of its sequence.

### EXAMPLE 3

Automatic Lengthening of the Partial Sequence

Automatic lengthening of partial sequence S is completed in three steps:

1. Determination of all sequences homologous to S from the total set of available sequences using BLAST

2. Assembling these sequences by means of the standard program GAP4 (Bonfield, J. K.; Smith, K. F. and Staden, R. (1995), Nucleic Acids Research 23 4992-4999) (contig formation)

3. Computation of a consensus sequence C from the assembled sequences.

The consensus sequence C will generally be longer than initial sequence S. Its electronic Northern Blot will accordingly deviate from that for S. A repeated Fisher test decides whether the alternative hypothesis of deviation from a uniform expression in the two libraries can be maintained. If this is the case, an attempt is made to lengthen C in the same way as S. This iteration is continued with consensus sequences Ci (i: iteration index) obtained in each case until the alternative hypothesis is rejected (if H, Exit; truncation criterion I) or until automatic lengthening is no longer possible (while Ci>Ci-1; truncation criterion II).

In the case of truncation criterion II, with the consensus sequence present after the last iteration, a complete or roughly complete sequence of a gene which can be related to cancer with high statistical certainty is acquired.

Analogously to the above-described examples, it was possible to find from ovarian tumor tissue the nucleic acid sequences Seq ID No. 115 (expression: overexpressed in ovarian tumor tissue; function: H. sapiens for neutrophil gelatinase associated lipocalin; module: lipocalin; cytogenetic localization: 9q34).

### EXAMPLE 4

Mapping of Nucleic Acid Sequences on the Human Genome

Human genes were mapped using the Stanford G3 Hybrid Panel (Stewart et al., 1997), which is marketed by Research Genetics, Huntsville, Ala. This panel consists of 83 different genomic DNAs of human-hamster hybrid cell lines and allows resolution of 500 kilobases. The hybrid cell lines were obtained by fusion of irradiated diploid human cells with cells of the Chinese hamster. The retention pattern of the human chromosome fragments is determined by means of gene-specific primers in a polymerasechain reaction and is analyzed using software available from the Stanford RH server (http://www.stanford.edu/RH/rhserver_form2.html). This program determines the STS marker that is nearest to the desired gene. The corresponding cytogenetic band was determined using the “Mapview” program of-the Genome Data-base (GDB), (http://gdbwww.dkfz-heidelberg.de).

In addition to mapping of genes on the human chromosome set by various experimental methods, it is possible to determine the location of genes on this by biocomputer methods. To do this, the known program e-PCR was used (Schuler GD (1998) Electronic PCR: Bridging the gap between genome mapping and genome sequencing. Trends Biotechnol 16: 456-459, Schuler GD (1997). Sequence mapping by electronic PCR. Genome Res. 7: 541-550). The database used here no longer corresponds to the one cited in the literature, but is a further development which includes data from the public database RHdb (http://www.ebi.ac.uk/RHdb/index.html). Analogously to the mapping by the hybrid panels, the results were evaluated with the above-mentioned software and the software of the Whitehead Institute (http://carbon.wi.mit.edu:8000/cgi-bin/contig/rhmapper.pl).

### EXAMPLE 5

Obtaining Genomic DNA Sequences (BAC Clones)

The genomic BAC clones that contain the corresponding cDNAs (http://www.tree.caltech.edu/; Shizuya, H.; B. Birren, U -J. Kim, V. Mancino, T. Slepak, Y. Tachiiri, M. Simon (1992) Proc. Natl. Acad. Sci., USA 89: 8794-8797) were isolated with the procedure of “down-to-the-well”. In this procedure, a library consisting of BAC clones (the library covers roughly 3× the human genome) is moved into a certain raster, so that the DNA of these clones with a specific PCR can be studied. In doing so, “pooling” of the DNA of different BAC clones takes-place. Combinatorial analysis makes it possible to determine the clones that contain the desired DNA. By fixing the clones, the address of the clones in the library can be determined. This address together with the name of the library which is being used unequivocally fixes the clones and thus the DNA sequence of these clones. The libraries used were CITB B and CITB C:

TABLE 1
Electronic Northern for Seq. ID No.: 115

| | NORMAL | TUMOR | Ratios | |
|---|---|---|---|---|
| | % frequency | % frequency | N/T | T/N |
| Bladder | 0.0039 | 0.0051 | 0.7627 | 1.3111 |
| Breast | 0.0000 | 0.0000 | undef | undef |
| Small intestine | 0.0016 | 0.0000 | undef | 0.0000 |
| Ovary | 0.0000 | 0.0702 | 0.0000 | undef |
| Endocrine tissue | 0.0000 | 0.0000 | undef | undef |
| Gastrointestinal | 0.0192 | 0.0185 | 1.0354 | 0.9658 |
| Brain | 0.0007 | 0.0000 | undef | 0.0000 |
| Hematopoietic | 0.0053 | 0.0000 | undef | 0.0000 |
| Skin | 0.0000 | 0.0000 | undef | undef |
| Hepatic | 0.0000 | 0.0000 | undef | undef |
| Heart | 0.0000 | 0.0000 | undef | undef |
| Testicles | 0.0058 | 0.0000 | undef | 0.0000 |
| Lung | 0.0052 | 0.0020 | 2.5402 | 0.3937 |
| Stomach-esophagus | 0.0193 | 0.0230 | 0.8404 | 1.1900 |
| Muscle-skeleton | 0.0000 | 0.0000 | undef | undef |
| Kidney | 0.0000 | 0.0000 | undef | undef |
| Pancreas | 0.0017 | 0.0110 | 0.1496 | 6.6857 |
| Penis | 0.0000 | 0.0000 | undef | undef |
| Prostate | 0.0065 | 0.0000 | undef | 0.0000 |
| Uterus-endometrium | 0.0000 | 0.0000 | undef | undef |
| Uterus-myometrium | 0.0000 | 0.0000 | undef | undef |
| Uterus-general | 0.0000 | 0.0954 | 0.0000 | undef |
| Breast hyperplasia | 0.0000 | | | |
| Prostate hyperplasia | 0.0000 | | | |
| Seminal vesicle | 0.0000 | | | |
| Sensory organs | 0.0118 | | | |
| White blood cells | 0.0000 | | | |
| Cervix | 0.0000 | | | |

| | | STANDARDIZED/ | |
|---|---|---|---|
| | | SUBTRACTED | |
| | FETUS | LIBRARIES | |
| | % frequency | % frequency | |
| Development | 0.0000 | Breast | 0.0000 |
| Gastrointestinal | 0.0028 | Ovary_n | 0.0000 |
| Brain | 0.0000 | Ovary_t | 0.0101 |
| Hematopoietic | 0.0000 | Endocrine tissue | 0.0000 |
| Skin | 0.0000 | Fetal | 0.0047 |
| Hepatic | 0.0000 | Gastrointestinal | 0.0122 |
| Heart-blood vessels | 0.0000 | Hematopoietic | 0.0114 |
| Lung | 0.0000 | Skin-muscle | 0.0065 |
| Suprarenal gland | 0.0000 | Testicles | 0.0000 |
| Kidney | 0.0000 | Lung | 0.0000 |
| Placenta | 0.0000 | Nerves | 0.0010 |
| Prostate | 0.0000 | Prostate | 0.0068 |
| Sensory organs | 0.0000 | Sensory Organs | 0.0000 |
| | | Uterus_n | 0.0167 |

1. A nucleic acid sequence that codes a gene product or a part thereof, comprising
a) a nucleic acid sequence Seq. ID No. 115,
b) an allelic variation of the nucleic acid sequence named under a) or
c) a nucleic acid sequence that is complementary to the nucleic acid sequence named under a) or b).

2. A nucleic acid sequence Seq. ID No. 115, characterized in that it is expressed elevated in ovarian tumor tissue.

3. BAC, PAC and Cosmid clones containing functional genes and their chromosomal localization according to sequence Seq. ID No. 115 for use as vehicles for gene transfer.

4. A nucleic acid sequence according to claim 1 or 2, wherein it has 90% homology to a human nucleic acid sequence.

5. A nucleic acid sequence according to claim 1 or 2, wherein it has 95% homology to a human nucleic acid sequence.

6. A nucleic acid sequence comprising a portion of the nucleic acid sequences named in claims 1 to 4, in such a sufficient amount that it hybridizes with the sequences according to the claims 1 to 5.

7. A nucleic acid sequence according to claims 1 to 5, herein the size of the fragment has a length of at least 50 to 4500 bp.

8. A nucleic acid sequence according to claims 1 to 5, wherein the size of the fragment has a length of at least 50 to 4000 bp.

9. A nucleic acid sequence according to one of the claims 1 to 8, which codes at least one partial sequence of a bioactive polypeptide.

10. An expression cassette, comprising a nucleic acid fragment or a sequence according to one of the claims 1 to 8, together with at least one control or regulatory sequence.

11. An expression cassette, comprising a nucleic acid fragment or a sequence according to claim 9, in which the control or regulatory sequence is a suitable promoter.

12. An expression cassette according to one of the claims 12 and 13, wherein the DNA sequences located on the cassette code a fusion protein, which comprises a known protein and a bioactive polypeptide fragment.

13. Use of nucleic acid sequences according to claims 1 to 9 for producing full-length genes.

14. A DNA fragment, comprising a gene, that can be obtained from the use according to claim 13.

15. Host cell, containing as the heterologous part of its expressible genetic information a nucleic acid fragment according to one of the claims 1 to 8.

16. Host cell according to claim 15, wherein it is a prokaryontic or eukaryontic cell system.

17. Host cell according to one of claims 17 or 18, wherein the prokaryontic cell system is E. coli and the eukaryontic cell system is an animal, human or yeast cell system.

18. A process for producing a polypeptide or a fragment, wherein the host cells according to claims 15 to 17 are cultivated.

19. An antibody that is directed against a polypeptide or a fragment that is coded by the nucleic acid of sequence Seq. ID No. 115, which can be obtained according to claim 18.

20. An antibody according to claim 19, wherein it is monoclional.

21. An antibody according to claim 19, wherein it is a phage display antibody.

22. Use of nucleic acid sequences according to claim 2 in a phage display process.

23. Use of the nucleic acid of sequence Seq. ID No. 115 for the expression of polypeptides that can be used as tools for finding active ingredients against ovarian cancer.

24. Use of the nucleic acid of sequence Seq. ID No. 115 in sense or antisense form.

25. A nucleic acid sequence according to claims 1 to 8, wherein it is a genomic sequence.

26. A nucleic acid sequence according to claims 1 to 8, wherein it is an mRNA sequence.

27. Genomic genes, their promoters, enhancers, silencers, exon structure, intron structure, and their splice variants, that can be obtained from cDNA of sequence Seq. ID No. 115.

28. Use of genomic genes according to claim 27, together with suitable regulatory elements.

29. Use according to claim 28, wherein the regulatory element is a suitable promotor and/or enhancer.

30. A nucleic acid sequence according to claims 1 to 5, wherein the size of the fragment has a lenght of at least 300 to 3500 bp.


SEQUENCE LISTING (1 sequence)

SEQ ID NO: 1 - DNA, 719 bp, homo sapiens
gtcgactttttttttttttttttaacatggaaaagtatttttaaaaatcgaataatcctattcaagtcaaccagtgttaaccccggtgtgcttcctgccagtctgttcctccccatgggagtcacacaaaatgaaaatctcctagaaagagaagacaaagacccgcaaaagatgtatgccaccatctatgagctgaaagaagacaagagctacaatgtcacctccgtcctgtttaggaaaaagaagtgtgactactggatcaggacttttgttccaggttgccagcccggcgagttcacgctgggcaacattaagagttaccctggattaacgagttacctcgtccgagtggtgagcaccaactacaaccagcatgctatggtgttcttcaagaaagtttctcaaaacagggagtacttcaagatcaccctctacgggagaaccaaggagctgacttcggaactaaaggagaacttcatccgcttctccaaatctctgggcctccctgaaaaccacatcgtcttccctgtcccaatcgaccagtgtatcgacggctgagtgcacaggtgccgccagctgccgcaccagcccgaacaccattgagggagctgggagaccctccccacagtgccacccatgcagctgctccccaggccaccccgctgatggagccccaccttgtctgctaaataaacatgtgccctcaaaaaaaaaaaaaa